(19) United States
(12) Patent Application Publication
(10) Pub. No.: US 2025/0242203 A1
(43) Pub. Date: Jul. 31, 2025

(54) Remote Fitness Monitoring Device Utilizing Facial Recognition and Photoplethysmography for Exercise Intensity and Health Tracking

(71) Applicant: James Buchheim, New York, NY (US)

(72) Inventor: James Buchheim, New York, NY (US)

(21) Appl. No.: 18/733,901

(22) Filed: Jun. 5, 2024

Related U.S. Application Data
(60) Provisional application No. 63/624,912, filed on Jan. 25, 2024.

(51) Int. Cl. A63B 24/00; A61B 5/00; A61B 5/022; A61B 5/024; A61B 5/1171

(52) U.S. Cl. CPC A63B 24/0062; A61B 5/0002; A61B 5/0033; A61B 5/022; A61B 5/02405; A61B 5/1176; A61B 5/4866; A61B 5/742; A63B 2024/0065; A63B 2220/17

(57) ABSTRACT

A system for remote monitoring of a workout, including: a processor; a non-transient memory; an imaging sensor; and a display device, the non-transient memory having computer-readable instructions stored thereon which, when executed by the processor, perform steps including: capturing imagery of at least a face of a subject, estimating and monitoring a peripheral blood volume pulse (BVP) or heart rate from the imagery using remote photoplethysmography (rPPG), performing facial age estimation using computer vision to analyze the imagery of the face to estimate an age of the subject, estimating a maximum heart rate using at least the age of the subject, calculating a heart rate zones as a predetermined percentage of the maximum heart rate, and displaying an indication of a current heart rate zone based on a current BVP or heart rate.

This patent application claims priority from, and the benefit of, U.S. Provisional Patent Application No. 63/624,912, filed Jan. 25, 2024, which is incorporated in its entirety as if fully set forth herein.

### FIELD OF THE INVENTION

The present invention relates to a system, device, and method for monitoring exercise intensity and for health tracking and, more particularly, to a system, device, and method tracking exercise intensity using various devices and methods, including using facial recognition and photoplethysmography from attached or remote devices.

### BACKGROUND OF THE INVENTION

Camera-based physiological measurement is a fast-growing field of computer vision. Remote photoplethysmography (rPPG) utilizes imaging devices (e.g., cameras) to measure the peripheral blood volume pulse (BVP) and enables cardiac measurement via webcams and smartphones.

Heart rate zones are based on a percentage of your maximum heart rate. The easiest way to calculate your max heart rate is using the age-adjusted formula 220−your age=max heart rate. Heart rate zones are essentially an indication of how hard your heart is working to pump your blood and keep up with the demands of what you're doing. The higher your heart rate, the higher the heart rate zone you're in and the harder your body is working to keep up.

### SUMMARY OF THE INVENTION

According to the present invention there is provided a system for remote monitoring of a workout, including: a processor; a non-transient memory; an imaging sensor; and a display device, the non-transient memory having computer-readable instructions stored thereon which, when executed by the processor, perform steps including: capturing imagery of at least a face of a subject, estimating and monitoring a peripheral blood volume pulse (BVP) or heart rate from the imagery using remote photoplethysmography (rPPG), performing facial age estimation using computer vision to analyze the imagery of the face to estimate an age of the subject, estimating a maximum heart rate using at least the age of the subject, calculating a heart rate zones as a predetermined percentage of the maximum heart rate, and displaying an indication of a current heart rate zone based on a current BVP or heart rate.

According to further features in preferred embodiments the method further detecting a gender of the subject based on computer vision analysis of the imagery of the face, wherein the maximum heart rate is further estimated based on the gender of the subject.

According to still further features a weight of the subject is estimated based on the estimated height of the subject and artifacts of the imagery of a body of the subject, analyzed using computer vision.

According to still further features the computer-readable instructions executed by the processor are further configured to estimate a height of the subject using a predefined height of the imaging sensor above a surface on which the subject is standing and computer vision analysis of the captured imagery.

According to still further features a weight of the subject is estimated based on the estimated height of the subject and artifacts of the imagery of the face of the subject analyzed using computer vision.

According to still further features a number of calories burned is estimated based on the heart rate zone and the weight.

According to still further features the processor is selected from the group including: a central processing unit (CPU), a Graphics processing unit (GPU), a combination CPU and GPU, a Tensor Processing Unit (TPU), a neural processing unit (NPU), and a combination of two or more thereof.

According to another embodiment there is provided a method for calculating a number of reps, a number of sets, and an interval time between the sets for a selected training goal, including: capturing imagery of a subject's workout; processing the imagery using image processing for monitoring a number of repetitions (reps) performed; detecting a beginning of a rest period; starting a first counter indicating a duration of the rest period for first type of training goal.

According to further features in preferred embodiments the first type of training goal is selected or is a default selection. According to still further features the method further includes starting a second counter at the same time as the first counter, the second counter indicating a duration of the rest period for a second type of training goal.

According to still further features the method further includes starting at least one additional counter at the same time as the first counter, the at least one additional counter indicating a duration of the rest period for at least one additional type of training goal.

According to still further features the type of training goal selected from the group including: weight loss, muscle building, endurance, strength, and a combination thereof.

According to another embodiment there is provided a method for monitoring usage of training equipment, the method including: providing one or more image sensors each having a field of view (FOV) of a workout area including one or more pieces of workout equipment; capturing images of a plurality of pieces of workout equipment; processing captured images to extract usage data of each of the plurality of pieces of workout equipment; and analyzing the extracted usage data to provide analytics regarding usage of each of the plurality of pieces of workout equipment.

According to further features, the method further includes a step of transferring raw image data from the one or more image sensors to a local or remote computer prior to the step of processing the captured images.

### BRIEF DESCRIPTION OF THE DRAWINGS

Various embodiments are herein described, by way of example only, with reference to the accompanying drawings, wherein:

FIG. 1 is a prior art simplified example block diagram 100 of models B, B+, A and A+ of Raspberry Pi™ devices;

FIG. 2 is a diagram of a device according to an aspect of the present invention;

FIG. 3 is an example pictorial diagram depicting an FOV of a camera of the monitoring device;

FIG. 4 is a flow diagram 400 of the process for detecting/calculating a heart rate zone;

FIG. 5 is a flow diagram 500 for calculating/estimating a calorie burn rate;

FIG. 6 is a flow diagram 600 of a method for storing and comparing a present session to one or more prior session;

FIG. 7 is an example device 700 according to an aspect of the present invention;

FIG. 8 is a flow diagram 800 of a process for indicating rest intervals;

FIG. 9 is a system according to another aspect of the invention;

FIG. 10 is a flow diagram of a process 1000 for analyzing equipment use.

### DESCRIPTION OF THE PREFERRED EMBODIMENTS

There is disclosed herein a system including one or more devices for remote monitoring of exercise intensity and/or health tracking. The term remote monitoring is used herein to denote the monitoring of a subject by one or more devices that are not physically attached to the subject. These devices are referred to herein as remote devices. It is noted that the remote monitoring may be absolute or only partial. That is to say that the remote monitoring may be accomplished solely by remote devices (absolute) or by a combination of remote devices and devices attached to the subject (partial).

One of the central aspects of the present method and system is the calculation of a heart rate zone (HRZ). A subject's heart rate zone indicates how hard the subject is exercising, and, accordingly, what body resources the subject is utilizing for energy. As a rule of thumb, the higher the heart rate gets, the more the body burns carbohydrates and protein for energy, and the less the body used fat for energy.

Heart rate zones are based on a percentage of the subject's maximum heart rate. The easiest way to calculate a maximum heart rate is by using the age-adjusted formula: 220−subject age=max heart rate. Heart rate zones are essentially an indication of how hard the heart is working to pump blood and keep up with the demands of what the body is doing. The higher the heart rate, the higher the heart rate zone and the harder the body is working to keep up.

However, there is more than one way of calculating the heart rate zone. Different people define these zones in different ways. Furthermore, the outcomes of being in the different heart rate zones may differ from person to person. Accordingly, the definitions that are discussed hereafter are approximate, when discussing parameters, and imprecise when discussing the outcomes of being in the different zones.

The aforementioned notwithstanding, the devices that sense and monitor the subjects during periods of exertion will be preprogrammed with exact parameters, based on the programmers' decision. So, there may be one device that indicates that a subject is, for example, in zone 4, when a different device indicates that the subject is in zone 5. This is not a fault in the detection mechanism but rather stems from different definitions/parameters with which the device is preprogramed. The same device will be consistent with the indication of zones, based on the programmed parameters.

Based on the above, it is made clear that the definitions of the zones (e.g., the point at which a subject stops burning fat and starts burning carbohydrates), the example formulas used to calculate when the heart rate is in which zone, and various other imprecise aspects/features are to be considered merely exemplary, and not limiting per se. In general, it is the hardware and/or software components and the interactions therebetween that define the present invention, not the parameter values.

One example definition identifies five heart rate zones based on fuel source and the “talking test”:
Zone 1: Approximately 85% of the calories being burned are fat. A person can easily hold a conversation.
Zone 2: Roughly 65% of the calories being burned are fat. One can still hold a light conversation in this zone, but may need to stop talking for a beat occasionally to take a breath.
Zone 3: About 45% of the calories being burned are fat. Talking in this zone takes effort.
Zone 4: No fat is being burned. One can talk at this point if absolutely necessary, but with great difficulty.
Zone 5: You can only keep up this amount of effort for a few minutes. Talking is not possible.

TABLE 1
Example of exercises and outcomes in the different zones.

| Zone | Also known as | Intensity level | % of max heart rate | Fuel source |
|---|---|---|---|---|
| Zone 1 | Warm-up, recovery, easy. | Moderate-low. | 50% to 60% | Fat. |
| Zone 2 | Aerobic, endurance, base, | Moderate. | 60% to 70% | Fat. |
| | light. | | | |
| Zone 3 | Tempo, threshold, cardio, | Moderate-high. | 70% to 80% | Fat, carbs, |
| | moderate. | | | protein. |
| Zone 4 | Lactate threshold, hard. | High. | 80% to 90% | Carbs, protein. |
| Zone 5 | Anaerobic, V02 max, | Very high. | 90% to 100% | Carbs, protein. |
| | maximum. | | | |

One example formula for calculating the heart rate zone (also known as training zone) is the Karvonen formula:

([Maximum heart rate−resting heart rate]×% intensity)+resting heart rate=training zone.

Another way of looking at this is:

$$\begin{matrix}1. & \\ Maximum heart rate - resting heart rate = heart rate reserve ( HRR ) & \end{matrix}$$

$$\begin{matrix}2. & \\ ( Heart rate reserve \times \% intensity ) + resting heart rate = training zone & \end{matrix}$$

Maximum Heart Rate—Maximum heart rate can be calculated in three ways: precisely, via testing; approximately, in an anecdotal manner; and with a mathematical formula.
Option 1: The gold standard for finding your maximal heart rate is a treadmill stress test in a lab.
Option 2: Use the highest heart rate observed during a race or high intensity workout in the last six months.
Option 3: 220 minus subject's age=estimate of max heart rate. This option can be reproduced without any prior knowledge of the subject and without any input from the subject.

A more precise calculation instead of option 3 would be:

$$Men : maximum heart rate = 241 - ( 0.8 \times age ) ;$$

$$Women : maximum heart rate = 209 - ( 0.7 \times age ) .$$

Resting Heart Rate—A subject's resting heart rate represents how many times their heart beats in a minute when they are sitting or lying still.

Heart Rate Reserve (HRR)—the difference between one's maximum heart rate and their resting heart rate.

The target heart rate for each heart rate zone is calculated by multiplying the heart rate reserve by the percentage of max heart rate that each zone represents: 50% (0.5) for zone 1; 60% (0.6) for zone 2; 70% (0.7) for zone 3; 80% (0.8) for zone 4; and 90% (0.9) for zone 5, according to some sources.

Other sources (including the American College of Sports Medicine) calculate the max heart rate by the following percentages per zone: zone 1 less than 57%; zone 2 57-63%; zone 3 64-76%; zone 4 77-95%; and zone 5 96-100%.

However the calculation is performed, it is clear that the following features need to be known for such a calculation: (1) a peripheral blood volume pulse (BVP); and (2) the subject's age. An additional feature that can be used is: (3) the subject's gender (see above for the more precise calculation of maximum heart rate based on age and gender). Camera-based physiological measurement is a fast-growing field of computer vision.

Age detection—remote age detection using computer vision is known in the art. Facial Age Estimation uses AI algorithms to estimate a user's age based on facial recognition. One example of machine learning models for age detection is known from Levi et al. “Age and Gender Classification using Convolutional Neural Networks” of the Department of Mathematics and Computer Science of The Open University of Israel. Gender detection is much more accurate, and is well known in the art.

Remote detection of a pulse is also known in the art, but likewise not sufficiently accurate. Remote photoplethysmography (rPPG) utilizes imaging devices (e.g., cameras) to measure the peripheral blood volume pulse (BVP) and enables cardiac measurement even via webcams and smartphones. One example publication is “rPPG-Toolbox: Deep Remote PPG Toolbox” by Liu et al. The toolbox is available online at: https://github.com/ubicomplab/rPPG-Toolbox. The toolbox contains unsupervised and supervised rPPG models with support for public benchmark datasets, data augmentation, and systematic evaluation.

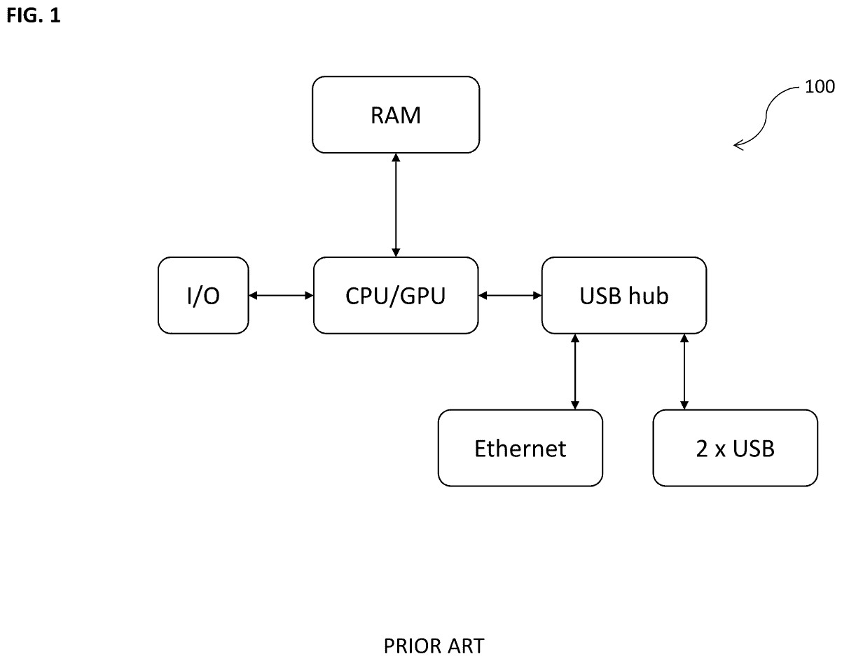

The hardware, firmware, and/or software for rPPG and age detection is incorporated/embodied in/on a computing device. For example, a Raspberry Pi™ device is a single-board computing device. FIG. 1 is a simplified example block diagram 100 of models B, B+, A and A+ of Raspberry Pi™ devices. The example device includes a CPU as well as a GPU. A different example from the same series, the Raspberry Pi™ 5, uses the Broadcom BCM2712 SoC (System on a Chip). The SoC features a quad-core ARM® Cortex®-A76 processor clocked at 2.4 GHz, alongside a VideoCore® VII GPU clocked at 800 MHz. The BCM2712 SoC also features support for cryptographic extensions. Alongside the new processor and graphics unit, the monolithic design of the earlier BCM2711 has been replaced with a CPU and chipset (southbridge) architecture, as the IO functionality has been moved to the Raspberry Pi 5's custom RP1 chip.

Obviously, these devices are merely examples of devices that can be used, or built onto, in order to provide the necessary hardware/firmware/software for the present invention. This or other computer boards are adapted to support audio and video input as well as output to a display peripheral. Examples are well known in the art.

In preferred embodiments, the device further includes an accelerometer. In embodiments, the device includes a tensor processing unit (TPU). A Tensor Processing Unit (TPU) is an AI accelerator application-specific integrated circuit (ASIC) (developed by Google®) for neural network machine learning.

In embodiments, the device includes a neural processing unit (NPU). An AI accelerator, deep learning processor, or neural processing unit (NPU) is a class of specialized hardware accelerator or computer system designed to accelerate artificial intelligence and machine learning applications, including artificial neural networks and machine vision. Typical applications include algorithms for robotics, Internet of Things, and other data-intensive or sensor-driven tasks. They are often manycore designs and generally focus on low-precision arithmetic, novel dataflow architectures or in-memory computing capability. As of 2024, a typical AI integrated circuit chip contains tens of billions of MOSFET transistors.

In embodiments, the device includes an image sensor such as a camera. In some embodiments, the device additionally, or alternatively, includes other non-contact vital sign monitoring systems such as, but not limited to, acoustic vibration, electromagnetic waves, and other methods. In some embodiments, the non-contact monitoring technologies are radar based. In embodiments, these non-contact monitoring systems may additionally, or alternatively, be configured or used to monitor or detect workout reps, heartbeat, hand movements, and in some cases even measure weight.

In some embodiments, the radar technology is mmWave (millimeter wave) radar technology. For example, recent research has proposed “a heart rate estimation scheme based on commodity off-the-shelf (COTS) millimeter-wave (mmWave) radar. The purpose is to suppress the interference of the random body motion, respiration, and its harmonics on the heartbeat signal at the software level and to improve the accuracy of heart rate estimation by analyzing the harmonic relationship of the heartbeat signal. The proposed signal processing algorithm consists of three parts. Firstly, a vital sign signal extraction algorithm is designed based on phase accumulation to suppress the Range Bin variation caused by random body motion and heavy breathing.

Adaptive Notch Filter (ANF) is combined with Empirical Wavelet Transform (EWT) to design a heartbeat signal extraction algorithm to suppress the interference of respiration and its harmonics. The respiratory harmonic components are suppressed by the ANF without affecting other features. EWT decomposes the composite signal into several bandlimited sub-signals. The final heart rate estimation is then performed based on the relationship of harmonics” Z. Ling, et al, “Non-Contact Heart Rate Monitoring Based on Millimeter Wave Radar,” in IEEE Access, vol. 10, pp. 74033-74044, 2022, doi: 10.1109/ACCESS.2022.3190355.

In some embodiments the device further includes a display device/peripheral. In embodiments, the device includes an accelerometer for sensing movement (e.g., exercise reps). In embodiments, the device is adapted to be worn on the body of the subject. In other embodiments, the device is placed on an exercise machine in order to detect movement of the machine (and in that way to count the reps).

However, it is most preferable that the device is positioned such that the body of the subject is within the field of view (FOV) for the camera so that the device can employ computer vision to remotely sense the subject's pulse (e.g., using rPPG), detect age, and possibly gender. With the age and pulse, the device/system can calculate the maximum heart rate and training zone, as discussed above. To this end, it is preferable that the device be placed on a stationary object (a wall, a pillar, a frame of an exercise machine) with an FOV of the exercise area.

In addition, the device can be further configured to recognize objects (using object detection and recognition features of computer vision) such as the type of exercise machine or the type of exercise accessory (e.g., weights, tension bands, skipping rope, etc.) the subject is using. Computer vision includes many additional sub-domains that are relevant to the instant system, such as, for example: event detection, activity recognition, video tracking, 3D pose estimation, learning, indexing, motion estimation, etc. The use of the aforementioned features will be discussed further with reference to rep counting, set counting and intervals between sets.

Another feature that is included in embodiments of the invention is calculating the number of calories being burned during the exercise session. In order for this calculation to be effective, it is necessary to further know the weight of the subject. As the instant system is intended to be usable even as a completely remote system, receiving no direct input from the subject and having no direct contact with the subject, it is necessary to calculate (or estimate, or a combination of both) the subjects weight using remote techniques only. To this end, the AI augmented system also captures the height of the subject and runs a “roundness” algorithm on the subject's face. Height is calculated by having the stationary height of the device preset in the system (i.e., how high off the ground the device has been placed, on the wall or pillar). Based on that preset constant value, the AI supported computer vision analyses the imagery of the subject captured by the device's camera to extrapolate the subject's height.

Furthermore, the image of the subject's face is processed using ML models to extrapolate a roundness of the face that is indicative of a weight of the subject (once the height of the subject is known). For example, a paper by Dantcheva et al. “Show me your face and I will tell you your height, weight and body mass index” was published in 2018 24th International Conference on Pattern Recognition (ICPR). The paper discusses estimating height, weight, and BMI from single-shot facial images by proposing a regression method based on the 50-layers ResNet-architecture. In addition, a novel dataset consisting of 1026 subjects was presented including results, which suggest that facial images contain discriminatory information pertaining to height, weight, and BMI, comparable to that of body-images and videos. The paper also includes a gender-based analysis of the prediction of height, weight and BMI.

Alternatively, or additionally, the imagery of the subject's body (preferably from more than one angle) can be processed by ML models and computer vision to extrapolate or estimate the weight of the subject. For example, a paper by Basit et al. titled “Human Weight Measurement Prediction with Visual Images with Artificial Neural Network Algorithm” was published in the Journal of Telematics and Informatics (JTI) Vol. 8, No. 1, March 2020, pp. 1-8.

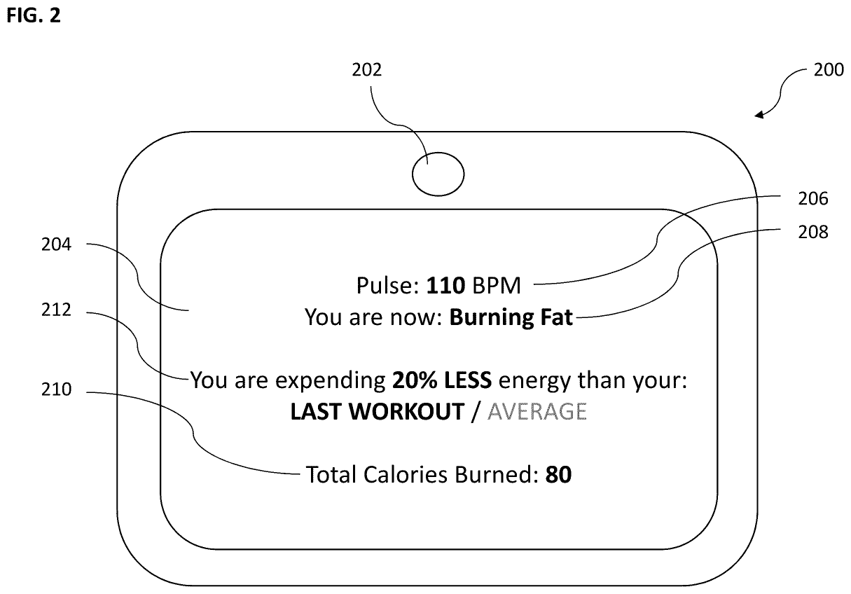

Bringing together the aforementioned components there is provided a device, method, and system for remotely estimating pulse, training zone, and/or calories burned. FIG. 2 is a diagram of a device according to an aspect of the present invention. In example embodiments the device is a compact computer or chipset (e.g., a Raspberry Pi® or similar device having similar components to those described above) 200, integrated with an imaging sensor, such as a regular camera 202, and in some cases Wi-Fi and/or alternative internet connectivity. In some embodiments, the device additionally, or alternatively, includes a different non-contact vital signs monitoring sensor, e.g., other types of optical imaging, acoustic vibration, electromagnetic waves, and other methods. In embodiments, the device includes a display interface, such as an onboard screen 204 that displays at least some of: the user's exercise intensity/training zone, real-time heart rate (pulse), calories burning per minute, and/or effort compared to previous sessions. For example, as shown in FIG. 2, the device displays a pulse rate of 110 BPM 206. In the depicted example embodiment, the device displays an indication of a heart rate zone, such as the fuel source being used (e.g., “burning fat”) 208 which is the result of being in heart rate zones 1 or 2 according to the version in Table 1 above. Another indication may be the type of training (e.g., “warm up”) or the intensity (e.g., “moderate”). The “training zone” can be indicated in many different ways, some of which have just been mentioned as examples. This indication can also/alternatively indicate a Training Goal. Training Goals may be any one of: weight loss, muscle building, endurance, strength, and/or a combination thereof.

In the example depicted embodiment, the device displays a number of calories burned 210. A calculation for estimating the number of calories burned is discussed elsewhere herein. In embodiments, the display interface may additionally be an input interface, such as a touch screen input/output device. In embodiments, the system may have different display options that can be selected via the I/O device (e.g., the touch screen display).

The system/device for remote monitoring of a workout includes a processor, a non-transient memory, an imaging sensor, and a display device. The non-transient/non-volatile memory has computer-readable instructions stored thereon which, when executed by the processor are configured to analyze captured imagery of a subject within the FOV of the imaging sensor. The device employs computer vision to detect and monitor a peripheral blood volume pulse (BVP) and calculate a heart rate zone. For example, implementing remote photoplethysmography (rPPG) to non-invasively measure heart rate through skin color changes detected by the camera. In order to calculate the heart rate zone (also referred to herein as a “training zone”, “intensity zone”, and variations thereof), it is necessary to perform Facial Age Estimation by detecting, extrapolating, and/or calculating an age of the subject, using computer vision/AI algorithms/ML models/Convolution Neural Networks (CNN)/Artificial Neural Networks (ANN) and the like to analyze the captured imagery of the subjects face. In embodiments, the calculation/estimation of the heart rate zone is further improved by detecting a gender of the subject, which improves the calculation/estimation of the subject's maximum heart rate.

In embodiments, the computer-readable instructions stored on the memory (software) and executed by the processor are further configured to estimate a height of the subject. The height being estimated based on the captured imagery and a predefined height at which the imaging sensor is disposed above a surface on which the subject is standing.

In embodiments, the software further calculates, extrapolates, and/or estimates a weight of the subject based on artifacts in the captured imagery of a face of the subject in addition to the height of the subject. In embodiments, the number of calories burned (e.g., per minute of the workout) is calculated based on the heart rate zone and the weight. In embodiments, the calculation is further improved based on the subject's gender, where the subject's gender is detected using computer vision to process imagery of the subject's face.

A user is recognized by the system either using indicia (e.g., inputting a code, sending an electronic signal [from a cellphone, RDIF tag, NFC circuit, magnetic strip of a card, etc.) or facial recognition.

Intensity of a workout is recorded during a workout session and stored in a database related to the user's file. The record or records may be stored locally on the device, or on a networked server connected to the devices, and/or stored remotely, e.g., on a cloud server, allowing the information to be retrieved even when not in the same location (e.g., the first workout is recorded in a gym and then the second workout is done in a different gym of the same chain of gyms, but in another city, or at home).

During the course of a workout, in some embodiments, the system compares current data regarding the training session to stored data. In embodiments, an indication of “relative exertion” 212, i.e., the aforementioned comparison is displayed on the display device. For example, the present session can be compared to a previous session, indicating whether the present session is better or worse than the previous session, such as displaying the message 212 “You are expending 20% LESS energy than your: LAST WORKOUT”. Such a comparison indicates that real-time (or near real-time) data is being compared to data of a previous session, but at a comparable stage of the workout session. In another example, the current session data is compared to an average of some, or all, of the previous sessions and an indication of that comparison is displayed on the device, such as: “You are expending 20% LESS energy than your: AVERAGE”. In some embodiments, the display device is a touch screen that allows the user to select which comparison to view.

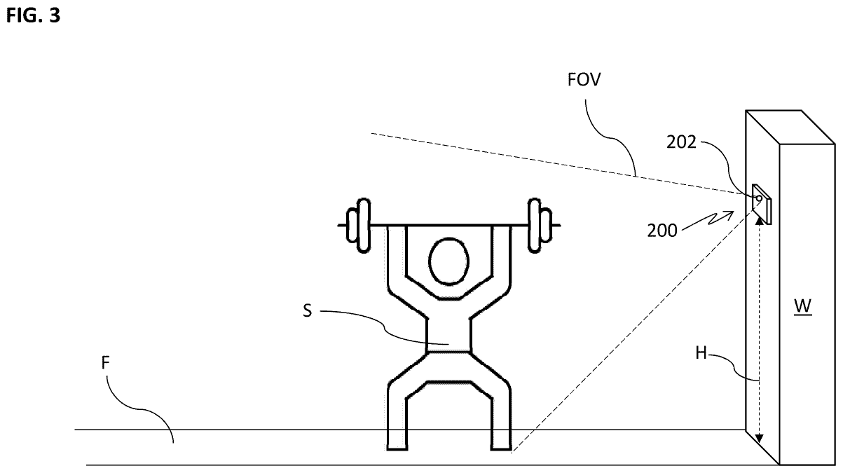

FIG. 3 is an example pictorial diagram depicting an FOV of a camera of the monitoring device. For example, monitoring device 200 is attached to a wall/pillar/post W at a known height H off the floor F. An example Field of View is depicted between two broken lines FOV stemming from the camera 202 of the monitoring device 200. The depiction is not necessarily to scale but rather to illustrate one example implementation of the device in a setting such as a gym. In the example depiction, a subject S is lifting weights. A subject's heart rate, height, and weight can be determined by processing images according to the scenario depicted in FIG. 3. Heart rate/BVP can be detected using rPPG as discussed elsewhere herein. Height can be determined based on the known height H of the camera and the captured imagery. Weight of the subject can be determined based on the height of the subject and facial artifacts detected in one or more images of the face of the subject as discussed elsewhere herein. Alternatively, or additionally, weight of the subject can be determined based on one or more images of the subjects' body, as discussed elsewhere herein.

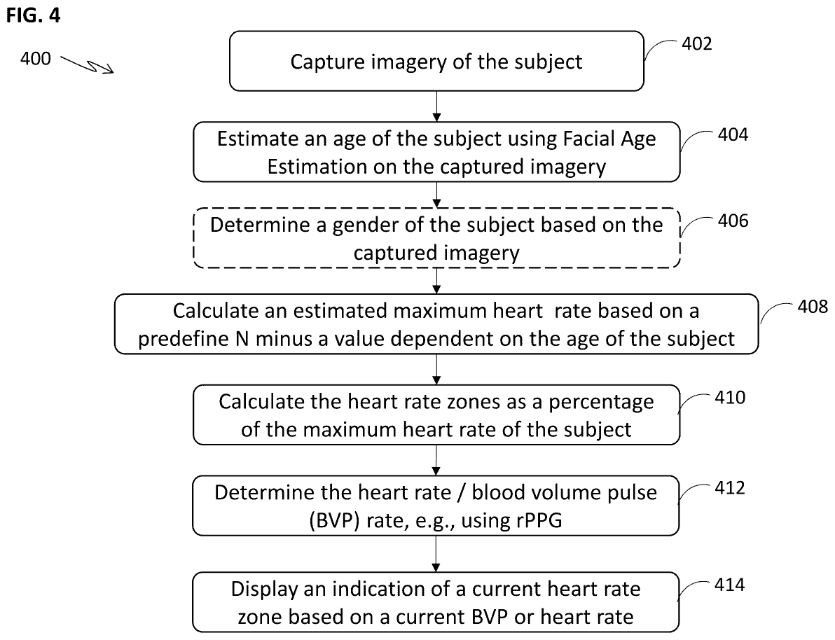

FIG. 4 is a flow diagram 400 of the process for detecting/calculating a heart rate zone. Some of the following steps may be made out of the order detailed hereafter, and some steps may be performed simultaneously or in parallel with other steps detailed hereafter. In step 402, the device starts to capture imagery of the subject. The images are stored on the device and subjected to various types of image processing. In step 404, one or more images are processed using computer vision including AI and ML models and or ANN/CNN to determine/estimate the age of the subject.

In step 406, an optional step, the images are processed to determine an age of the subject. In step 408 an estimated maximum heart rate of the subject is calculated using the formula of a predetermined constant N minus a value dependent on the age of the subject. As discussed elsewhere herein, one formula has N=220 and the value dependent on the age is the age number itself. According to another formula, if the subject is a male, then N=214 and the age-dependent value is 0.8×age number. If the subject is female, then N=209 and the age-dependent value is 0.7×age number.

In step 410 the heart rate zones/training zones are calculated using predetermined parameters where the zones are determined as a percentage (exactly what percentage is predetermined) of the calculated maximum heart rate of the subject.

In step 412, the captured imagery is subjected to real-time or near-real-time processing in order to determine the heart rate/blood volume pulse (BVP) rate, using, for example, rPPG to non-invasively, and without contact, measure heart rate through skin color changes detected by the camera. In some embodiments, the device additionally, or alternatively, includes other non-contact vital sign monitoring systems such as, but not limited to, acoustic vibration, electromagnetic waves, and other methods. In some embodiments, the non-contact monitoring technologies are radar based. In embodiments, these non-contact monitoring systems may additionally, or alternatively, be configured or used to monitor or detect workout reps, heartbeat, hand movements, and in some cases even measure weight.

In step 414, display an indication of a current heart rate zone based on a current BVP or heart rate. In some embodiments, there is no display, rather the sensor data/detected telemetry data (e.g., the workout data, including the detected BVP or heart rate data, etc.) is recorded and communicated to a central and/or remote network computer and/or to a personal computing device such as a smartphone or smartwatch/wearable. In some embodiments, the data is displayed, stored, and/or communicated as above.

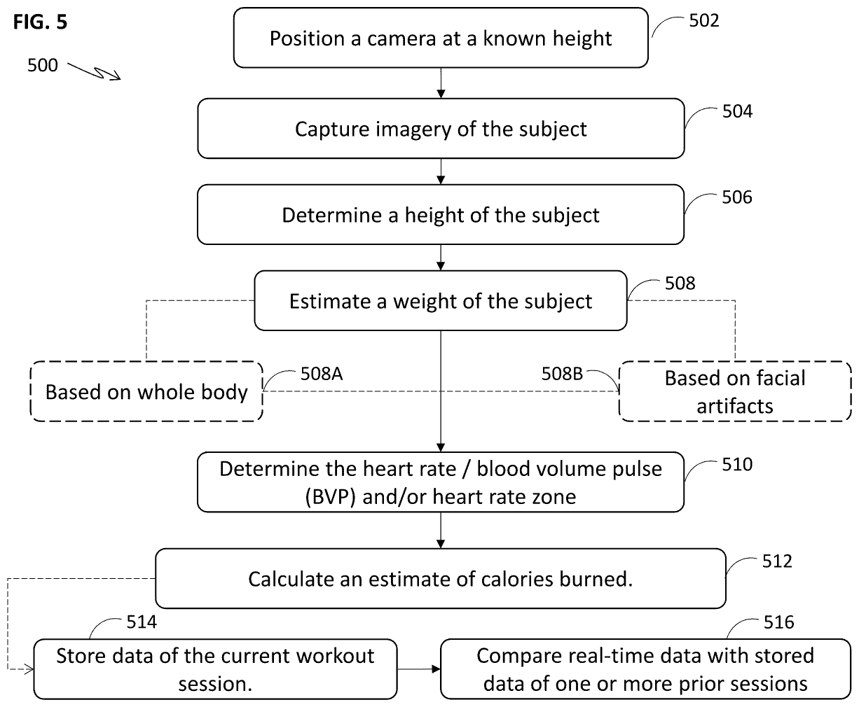

FIG. 5 is a flow diagram 500 for calculating/estimating a calorie burn rate. At step 502, a device according to the present invention is set at a known/predetermined height of the ground.

At step 504, imagery of a subject is captured.

At step 506, the height of the subject is calculated based on the known/predetermined height and the captured imagery of the subject.

At step 508, a weight of the subject is estimated. In embodiments, the subject's weight is estimated based on one or more captured images of the entire body of the subject 508A which are processed using image processing and trained ML models used for the prediction of weight of a person.

In embodiments, one or more images of the face of the subject 508B that were captured are processed using image processing and trained ML models used for the prediction of weight of a person based on facial artifacts. In embodiments, the weight is estimated based on both of the aforementioned body and face calculations.

At step 510, determine a current heart rate and/or a heart rate zone.

At step 512, calculate an estimate of calories burned based on height and/or weight and current heart rate and/or heart rate zone. Methods for calculating the number of calories burned are known in the art. In general, the more information known about the subject, the more accurate the calculation/estimate will be. One example formula is METS×3.5×BW (KG)/200=KCAL/MIN.

METS are metabolic equivalents which are defined as caloric consumption (by means of breathing) of an active individual compared with their resting basal metabolic rate (i.e., the total number of calories burned when the body is completely at rest). It is based on how much oxygen the body consumes during activity compared to how much oxygen the body consumes at rest. Most individuals at rest utilize 3.5 mL of oxygen per kg of body weight per minute, which equates to 1 kcal/kg/hr. However, it is noted that this value was pre-determined by testing on a 40-year-old man weighing 70 kg. More recent studies show that true resting MET values are much lower than 3.5 mL/kg/min and that age, gender, body fat, muscle mass, and illness significantly affect this value. However, this equation is used to provide people with estimates.

Therefore, it can be assumed that 1 MET is the equivalent to a Vo2 (a measure of oxygen consumption) of 3.5 mL/kg/min (resting state) and is equal to burning 1 kcal/min. If something requires 2 METS, then it requires twice the resting metabolism. When performing exercise, MET values are assigned to various forms of physical activity to determine how many calories are expended during that activity. For example, running at a speed of 7 mph (8.5 min/mile) is assigned the value: 11.5.

According to the formula above, a 30-year-old man weighing 170 lbs (77.3 kg) performs 45 minutes of running at 7 mph, then the amount of calories he would burn per minute would be:

$$11.5 ( 3.5 ) ( 77.3 kg ) / 200 = 15.6 kcals / min$$

So, in 45 minutes, this man would burn 700 calories running at 7 mph.

According to some embodiments, the device further employs image processing with AI/ML trained models to analyze captured video images of the subject to determine what kind of exercise activity is being performed and calculate the calorie consumption based, inter alia, on the MET of the detected activity.

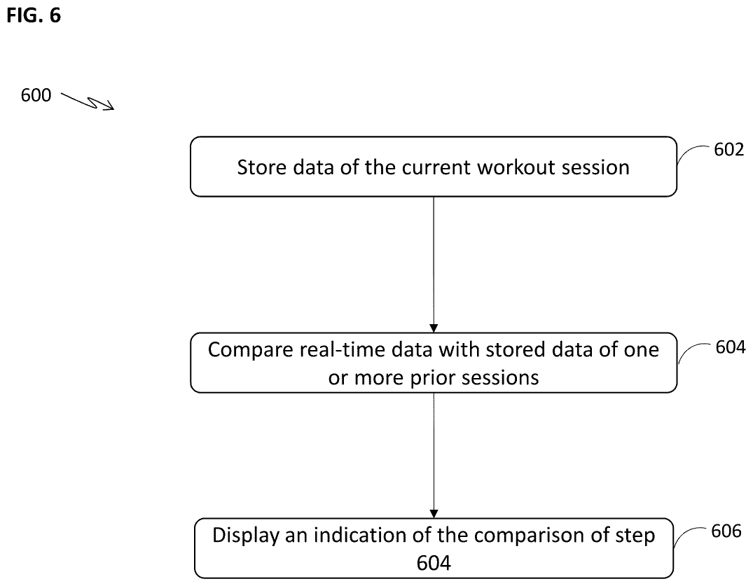

In some embodiments, the method further includes storing session data and comparing current session data to previous sessions. FIG. 6 illustrates a flow diagram 600 of a method for storing and comparing a present session to one or more prior session.

At step 602, the system stores session data of a current workout session in real-time (or near real-time).

At step 604, compare current session data to data of one or more previous sessions stored for the same subject.

At step 606, display an indication of the comparison performed in step 604.

There is disclosed herein a system and method for calculating a number of reps, a number of sets, and an interval time (also referred to as ‘rest periods’ and ‘inter-set rest’) between the sets for a selected Training Goal/Training Type. ‘Reps’ is the abbreviation used for the term ‘repetitions’, denoting a single execution of an exercise. One pushup is one rep, and 10 pushups are 10 reps. The term “set” or “sets” refers to is a collection of reps. If a goal is to complete 20 pushups, one might break the workout up into two sets of 10 reps. A set may include a plurality of reps of different activities, e.g., 10 pushups, 10 sit-ups, 10 squats may together constitute a set. Sets are spaced apart by intervals or rest periods. Muscles use specific energy systems for very short-duration or high-force activities. The rest periods between sets of resistance training can be changed to achieve certain Training Goals, such as: weight loss, muscle building, strength, or endurance. In embodiments where the device includes a display, an indication of a Training Goal based on the rest period between sets can be displayed.

For example, in hypertrophy training (muscle building), the objective is to overload the muscles and cause temporary trauma to the muscle fibers so that they are stimulated to grow and increase their cross-sectional area. Typically, there is very little difference between the loads handled for those wanting to induce hypertrophy and those wishing to solely increase strength. These loads typically range from 50%-90% of the subject's maximum for one rep (repetition). However, the biggest difference in training for muscle size versus strength is in rest between sets. Studies (e.g., “Short inter-set rest blunts resistance exercise-induced increases in myofibrillar protein synthesis and intracellular signalling in young males” by McKendry et al., published in Experimental Physiology, Volume 101, Issue 7, 1 Jul. 2016 Pages 866-882) have found that to induce muscle hypertrophy, optimal rest intervals are between 30-90 seconds.

According to Suchomel et al., in “The Importance of Muscular Strength: Training Considerations” (Sports Med. 2018 April; 48(4):765-785. doi: 10.1007/s40279-018-0862-z. PMID: 29372481), the typical rest periods for increasing strength are between 2-5 minutes, which research shows to be optimal for strength development. However, researchers note that this may vary depending on age, fiber type, and genetics.

According to other sources, the interval times are broken down as follows: (1) 20-60 seconds for fat burn; (2) 1-2 minutes for muscle growth; (3) 2-3 minutes for muscle growth and strength; and (4) 3-5 minutes for strength and power.

As can be seen from above, the exact parameter values are completely agreed on. However, it is clear that some given set of parameters will be programmed into the system that will be detailed hereafter. Therefore, different systems may give different results/outputs if the set of parameters are different, but the same system will give consistent results for different users, based on whatever set of parameters has been chosen and programmed into the system.

An individual's capacity to complete a high volume of Recovery Exercise within a given training session is inversely related to the average amount of rest taken between sets. This relationship is primarily due to the fact that shorter rest periods allow the body less time to clear function-reducing metabolites such as carbon dioxide and free hydrogen from muscle. Rationale for the development of individualized rest periods based on a real-time indicator of fatigue (HR) is such that this method would enable exercisers to adjust between-set rest intervals on a set-by-set basis, thereby preventing early cessation of exercise due to unmanageable levels of metabolic accumulation. By increasing the number of sets in which a target number of repetitions is completed, individuals may be able to increase their total training volume, which may in turn lead to greater hypertrophy if continued over the course of an appropriately-designed multi-week training program.

According to embodiments, the interval system can use the device and system detailed above, or a similar computer system. At least the device includes a computer (at least as detailed above and elsewhere herein), a camera, and a display. In some embodiments, the device may further include a Human Machine Interface (HMI), such as a touchscreen display.

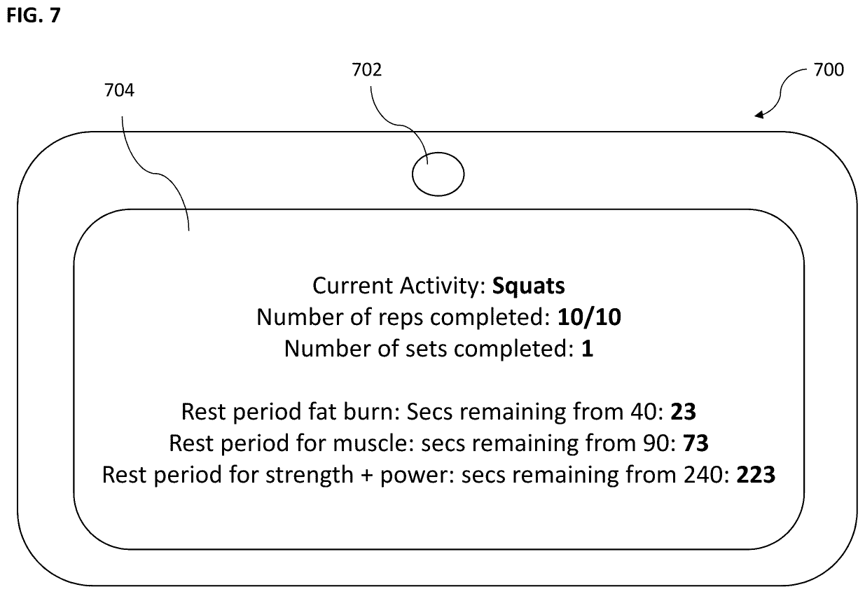

FIG. 7 illustrates an example device 700 according to an aspect of the present invention. Device 700 includes a computer having a processing unit and non-transient/non-volatile memory having stored thereon computer-readable instructions which, when processed by the processing unit, perform at least the process/steps detailed hereafter. The device 700 includes a camera or other non-contact sensor 702 and a display 704. According to some embodiments, display 704 is both an output device as well as an input device, such a touchscreen I/O component. In some embodiments, there is no display, rather the sensor data/detected telemetry data (e.g., the workout data, including the detected BVP or heart rate data, etc.) is recorded and communicated to a central and/or remote network computer and/or to a personal computing device such as a smartphone or smartwatch/wearable. In some embodiments, the data is displayed, stored, and/or communicated as above.

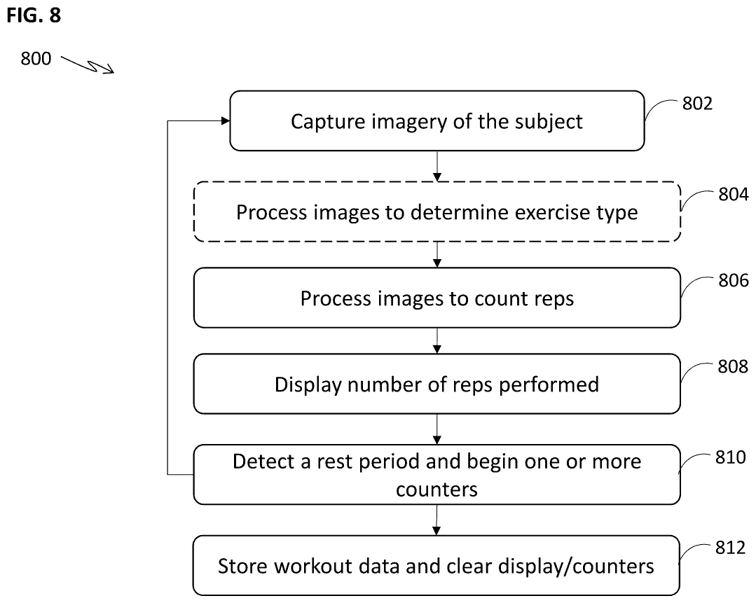

Using this device/system, the process includes a number of steps. FIG. 8 is a flow diagram 800 of a process for indicating rest intervals. At step 802 the camera captures images of the subject or another non-contact sensor captures data/telemetry from the subject's workout (as discussed elsewhere herein). At step 804 the images are processed to determine a type of activity being performed by the subject. In some embodiments, this step is optional/skipped. Computer vision techniques for event detection and/or activity recognition may be used to determine the type of exercise the subject is performing. In embodiments, the type of exercise is displayed on the screen (e.g., on display 704 in FIG. 7 “Current Activity: Squats”).

At step 806, the system continually processes the images to determine how many repetitions (reps) of the exercise have been performed. In embodiments, the number of reps is displayed on the screen at step 808. In the example illustration of FIG. 7 the screen displays the message: “Number of reps completed: 10/10”, indicating that the computer program, e.g., using AI/trained ML models, has determined that a set consists of 10 reps and that 10 reps have been detected. The AI/ML model predicts that the set has been completed and the next set will also include 10 reps.

At step 810, the AI/ML model/image processing detects that the subject has begun a rest period and begins one or more counters indicating the remaining time until the rest period/interval ends. In some embodiments, one counter is displayed (e.g., a fat burning counter, such as the counter “Rest period fat burn: Secs remaining from 40: 23” displayed in FIG. 7), based on a previous selection by the subject or a preset configuration.

In some embodiments, two or more counters begin simultaneously, each indicating a different exercise or Training Goal. In FIG. 7, three example counters are displayed: (1) “Rest period fat burn: Secs remaining from 40: 23”; (2) “Rest period for muscle: sees remaining from 90: 73”; and (3) “Rest period for strength+power: sees remaining from 240: 223”. To clarify, for example, counter (1) indicates that a rest period for burning fat is 40, taking the average of the range quoted above between 20-60 seconds. In the example, 17 seconds have elapsed since the beginning of the rest period. Similarly, counter (2) denotes a rest period suggested to increase muscle building. The counter (2) starts at 90 seconds, which is the average of the range quoted above between 1-2 minutes. The same 17 seconds have elapsed, leaving 73 seconds until the end of the rest period (if the goal of the workout is to increase muscle size). The same explanation holds true for counter (3).

If the user begins the next set, then the process continues at step 802. The rest counters are stopped. The rep counter is reset. The new images a processed to ascertain if a same or different activity is being performed, etc.

If the workout is completed, then the process moves to step 812. At step 812 the session data is stored for the user and the counters and display messages reset.

According to some embodiments, by detecting the users heart rate or Pulse, the device can recommend how long to wait for better results from each set. For example, building muscle it is required to wait longer in between sets. For losing weight, it is required to wait less, maintaining the heart rate in the fat burn zone. According to embodiments, the device recognizes that the user has finished a set, and starts one or more count-down timers to start the next set. For example, one timer shows the countdown to stay in the fat burning zone, based on a predefined parameter, by acquiring a pulse via rPPG, and/or based on a combination of the two. In the example, another timer shows the countdown to build muscle, based on a predefined parameter and/or by acquiring the pulse via rPPG, and calculating the proper rest based on a relevant formula known in the art.

According to such embodiments, the heart rate of the subject is measured during the rest period. The system calculated the Training Goal based on the HR and rest period and updates the Training Goal based on the heart rate of the subject and the duration of the rest period.

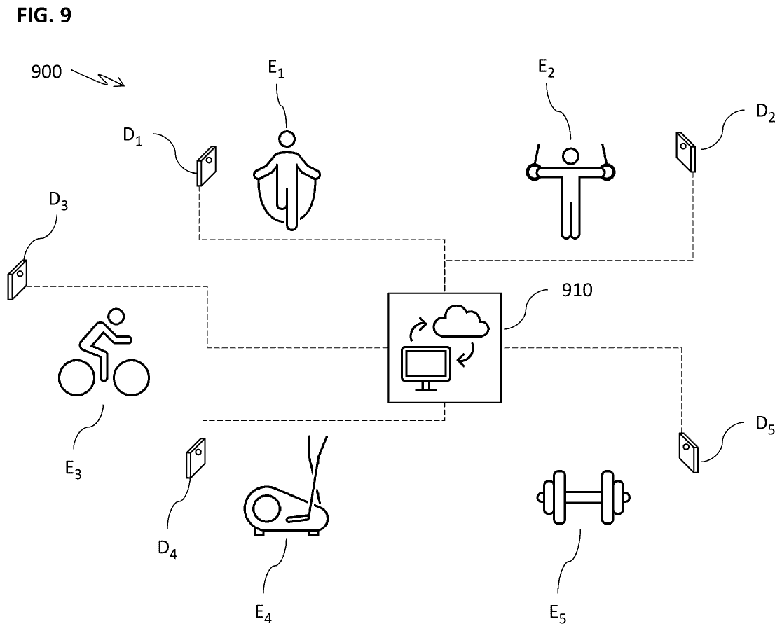

FIG. 9 illustrates a system according to another aspect of the invention. In FIG. 9 there is provided a computer system 900 for personalized tracking/monitoring of training on equipment, e.g., within a gym. There is provided a plurality of devices D_{1.5} for monitoring use of a plurality of exercise machines and exercise equipment E_{1.5} (hereafter referred to collectively as ‘equipment’, ‘pieces of equipment’, and variations thereof).

The devices D_{1.5} are linked to a central computing device via wired or wireless connections. In some embodiments, the computer system is a localized network coupled to a central computer. In other embodiments, the system additionally, or alternatively, includes a cloud server/network. Other configurations are well known in the art. According to any of the configurations, each of the devices monitors a machine or exercise area. Cameras on the devices capture images within the FOV and onboard or remote computing software processes the images to extract pertinent data. In embodiments, the individual devices are equipped with hardware and software employing AI/ML models (e.g., artificial neural networks, convolution neural networks, trained machine learning models, artificial intelligence, etc.) to extract information such as: how many people use a particular machine or equipment, for how long, what type of exercises are performed with the equipment, a demographic breakdown of the equipment users etc. The extracted data (and in some cases the raw image data) is transferred to the central computer/cloud computer for storing and analysis.

In other embodiments, the devices are simpler and merely record the FOV and transfer the image data to a central computer or to the cloud for further processing. In such a scenario/configuration, the image processing described heretofore is performed in the central/cloud computer. In all embodiments, the extracted data is analyzed, and the analytics are used by the gym for optimization of equipment and other resources.

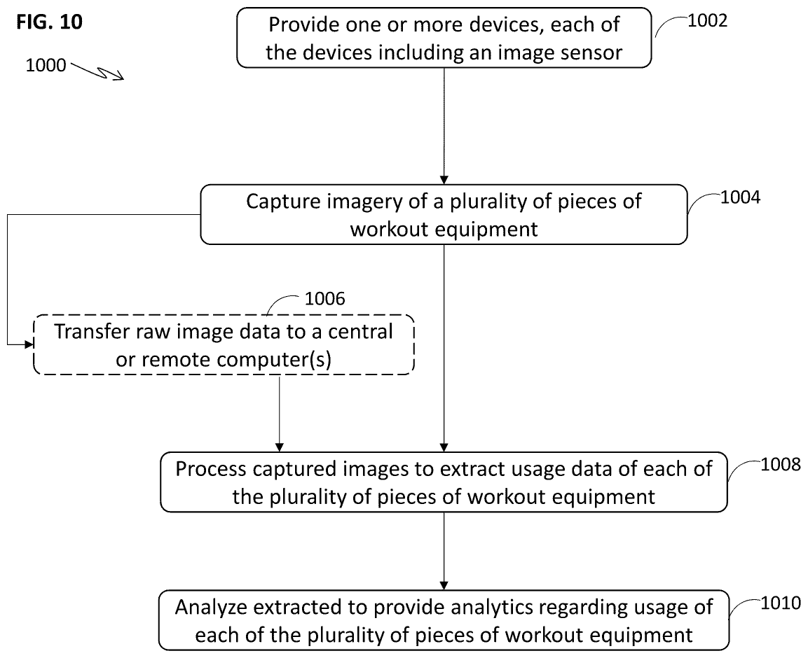

Based on the above, there is provided herein a method for analyzing equipment use. FIG. 10 illustrates a flow diagram of a process 1000 for analyzing equipment use. In step 1002, one or more devices (e.g., devices D_{1.5} in FIG. 9) are provided, each of the devices including an image sensor (e.g., cameras of devices D_{1.5}). The one or more image sensors each has a field of view (FOV) of a workout area including one or more pieces of workout equipment. The one or more image sensors capture images of a plurality of pieces of workout equipment.

In step 1004, images are captured by each of the one or more image sensors (e.g., cameras of devices D_{1.5}) of a respective FOV thereof, capturing images of the plurality of pieces of workout equipment. In step 1008, the captured images are processed to extract usage data of each of the plurality of pieces of workout equipment.

In step 1010, the extracted usage data is analyzed to provide analytics regarding usage of each of the plurality of pieces of workout equipment. In some embodiments, the processing is performed on the device that captures the images. In other embodiments, the processing is performed on a central or remote computing device. According to the latter embodiment, in step 1006, the captured image data is sent to the central or remote computer prior to processing step 1008. Based on the analytics, a gym can propose various customized offers and targeted advertising to members based on their activities in the gym. For example, a premium tier can be added for customers that stay longer.

According to other aspects of the invention, there is disclosed herein a method and system for user identification, including anonymous user identification, as well as data management. User identification, using image processing, is well known in the art, and can be established in many ways. One well known way is facial recognition. There is disclosed herein a system and method which allows for identification of the user even without storing any user information, especially not biometric data. According to embodiments of the invention, Anonymous User Identification entails the generation of a random Unique User Identification (UUID) based on facial detection characteristics. Using only these markers ensures privacy, as no actual facial images are stored on the system or transmitted between devices. Using one or more of the systems detailed above, a user can be identified as an anonymous user with a UUID under which all of the user's data is stored (e.g., on a local network and/or cloud). The data can include the recorded workout telemetry of the vital signs (heart rate, blood pressure, respiration etc.) as well as the recorded workout data (as discussed above, such as, training zones, training goals, number or reps and sets etc.).

A system, such as, for example, the system depicted in FIG. 9 can include multiple devices within a facility (e.g., gym) and be configured to recognize a user's UUID and display personalized workout data, including comparisons with previous workouts (as discussed above with regards to FIG. 2, the details of which are to be seen as if repeated here, mutatis mutandis). In embodiments, the system uses facial recognition to recognize a user and count and display, for the recognized user, a number of sets performed on a selected machine, even after one or more intervals between sets when the selected machine was not used by the user.

With regards to any and all of systems disclosed herein, there are various data storage options (e.g., local network, cloud computing, serverless computing, etc.). The various options afford flexibility to store workout data on a local server or a cloud-based platform.

Alternatively, or additionally, there are enhanced user engagement features, that can be integrated with any of the systems disclosed above. Features include, for example, QR Code and/or Bluetooth Integration whereby users can scan the device with a custom app to provide additional personal data (gender, height, weight), enabling more accurate calculations of calorie burn rate, by providing weight, height and/or gender as needed, and displaying them either on the screen on the device, or within the user's app in their phone. Another feature entails every device having a barcode/QR code, etc., so that a user can store details on the cloud and on the app. This information is stored for the user, and has all the details of machine, pulse rate, reps and sets.

Another feature is Comprehensive Workout Analysis whereby the device, in conjunction with the app, provides detailed workout summaries, including target zones, calories burned per minute, workout duration, and exercise intensity zones.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, non-transitory storage media such as a magnetic hard-disk and/or removable media, for storing instructions and/or data. Optionally, a network connection is provided as well. A display and/or a user input device such as a keyboard or mouse are optionally provided as well.

For example, any combination of one or more non-transitory computer readable (storage) medium(s) may be utilized in accordance with the above-listed embodiments of the present invention. A non-transitory computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable non-transitory storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

As will be understood with reference to the paragraphs and the referenced drawings, provided above, various embodiments of computer-implemented methods are provided herein, some of which can be performed by various embodiments of apparatuses and systems described herein and some of which can be performed according to instructions stored in non-transitory computer-readable storage media described herein. Still, some embodiments of computer-implemented methods provided herein can be performed by other apparatuses or systems and can be performed according to instructions stored in computer-readable storage media other than that described herein, as will become apparent to those having skill in the art with reference to the embodiments described herein. Any reference to systems and computer readable storage media with respect to the following computer-implemented methods is provided for explanatory purposes and is not intended to limit any of such systems and any of such non-transitory computer-readable storage media with regard to embodiments of computer-implemented methods described above. Likewise, any reference to the following computer-implemented methods with respect to systems and computer-readable storage media is provided for explanatory purposes and is not intended to limit any of such computer-implemented methods disclosed herein.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware—based systems that perform the specified functions or acts, or combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein, the singular form “a”, “an” and “the” include plural references unless the context clearly dictates otherwise.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The above-described processes including portions thereof can be performed by software, hardware and combinations thereof. These processes and portions thereof can be performed by computers, computer-type devices, workstations, processors, micro-processors, other electronic searching tools and memory and other non-transitory storage-type devices associated therewith. The processes and portions thereof can also be embodied in programmable non-transitory storage media, for example, compact discs (CDs) or other discs including magnetic, optical, etc., readable by a machine or the like, or other computer usable storage media, including magnetic, optical, or semiconductor storage, or other source of electronic signals.

The processes (methods) and systems, including components thereof, herein have been described with exemplary reference to specific hardware and software. The processes (methods) have been described as exemplary, whereby specific steps and their order can be omitted and/or changed by persons of ordinary skill in the art to reduce these embodiments to practice without undue experimentation. The processes (methods) and systems have been described in a manner sufficient to enable persons of ordinary skill in the art to readily adapt other hardware and software as may be needed to reduce any of the embodiments to practice without undue experimentation and using conventional techniques.

While the invention has been described with respect to a limited number of embodiments, it will be appreciated that many variations, modifications and other applications of the invention may be made. Therefore, the claimed invention as recited in the claims that follow is not limited to the embodiments described herein.

1. A system for remote monitoring of a workout, comprising:
a device including: a processor; a non-transient memory; and an imaging sensor or other non-contact vital signs sensor, the non-transient memory having computer-readable instructions stored thereon which, when executed by the processor, perform steps including:
capturing imagery of at least a face of a subject,
estimating and monitoring a heart rate or peripheral blood volume pulse (BVP) from the imagery using remote photoplethysmography (rPPG),
performing facial age estimation using computer vision to analyze the imagery of the face to estimate an age of the subject,
estimating a maximum heart rate using at least the age of the subject,
calculating a Heart Rate Zone as a predetermined percentage of the maximum heart rate;
the device further including or is in communication with wireless communications components and wherein the device is configured to send captured data of the user to a remote network and wherein facial data of the subject is not stored on the device and is hidden behind a hashed unique user identification (UUID) on the remote network.

2. The system of claim 1, further including a display, and displaying an indication of the calculated heart rate zone.

3. The system of claim 1, further including a display, and displaying an indication of a Training Goal based on the calculated heart rate zone.

4. The system of claim 1, wherein the device further includes wireless communications components and wherein the device is configured to send captured data to a central computer or a remote network or the central computer and remote network.

5. (canceled)

6. The system of claim 4, wherein the central computer or remote network communicates data to a user device including at least one of: a Training Goal, a heart rate zone, a comparison to one or more previous workouts, a number of reps, and a number of sets.

7. The system of claim 1, further detecting a gender of the subject based on computer vision analysis of the imagery of the face, wherein the maximum heart rate is further estimated based on the gender of the subject.

8. The system of claim 1, wherein a weight of the subject is estimated based on an estimated height of the subject and visual indicators from imagery of a body of the subject, analyzed using computer vision.

9. The system of claim 1, wherein the computer-readable instructions executed by the processor are further configured to estimate a height of the subject using a predefined height of the imaging sensor above a surface on which the subject is standing and computer vision analysis of the captured imagery to provide an estimated height.

10. The system of claim 9, wherein a weight of the subject is estimated based on the estimated height of the subject and visual indicators from the imagery of the face of the subject analyzed using computer vision.

11. The system of claim 10, wherein a number of calories burned is estimated based on the heart rate zone and the weight.

12. The system of claim 1, wherein the processor is selected from the group including: a central processing unit (CPU), a Graphics processing unit (GPU), a combination CPU and GPU, a Tensor Processing Unit (TPU), a neural processing unit (NPU), and a combination of two or more thereof.

13. A method for automatically calculating a number of reps performed, a number of sets completed, and an interval time between the sets for Training Goals, comprising:
capturing imagery of a subject's workout;
processing the imagery using image processing for monitoring a number of repetitions (reps) performed;
detecting a beginning of a rest period;
starting a first counter indicating a duration of the rest period for first type of Training Goal.

14. The method of claim 13, wherein the first type of training goal is selected or is a default selection.

15. The method of claim 13, further comprising:
starting a second counter at the same time as the first counter, the second counter indicating a duration of the rest period for a second type of training goal.

16. The method of claim 15, further comprising:
starting at least one additional counter at the same time as the first counter, the at least one additional counter indicating a duration of the rest period for at least one additional type of training goal.

17. The method of claim 13, wherein the type of training goal selected from the group including: weight loss, muscle building, endurance, strength, and a combination thereof.

18. The method of claim 13, further comprising: counting a number of sets.

19. The method of claim 13, further including using facial recognition to recognize a user and count and display, for the recognized user, a number of sets performed on a selected machine, even after one or more intervals between sets when the selected machine was not used by the user.

20. The method of claim 13, further comprising:
measuring a heart rate of the subject;
updating the Training Goal based on the heart rate of the subject and the duration of the rest period.

21. (canceled)

22. (canceled)

23. The system of claim 4, wherein the central computer or the remote network or the central computer and remote network are configured to perform one or more of: estimating and monitoring a heart rate or BVP, performing the facial age estimation, estimating the maximum heart rate, calculating the Heart Rate Zone, providing a Training Goal, providing a comparison to one or more previous workouts, a number of reps, and a number of sets.
